(19) United States
(12) Patent Application Publication
(10) Pub. No.: US 2021/0198605 A1
(43) Pub. Date: Jul. 1, 2021

(54) INFORMATION PROCESSING APPARATUS, INFORMATION PROCESSING METHOD, PROGRAM, AND OBSERVATION SYSTEM

(71) Applicant: SONY CORPORATION, TOKYO (JP)

(72) Inventor: TAKESHI OHASHI, KANAGAWA (JP)

(21) Appl. No.: 16/757,192

(22) PCT Filed: Oct. 4, 2018
(86) PCT No.: PCT/JP2018/037145

§ 371 Date: Apr. 17, 2020

(30) Foreign Application Priority Data
Oct. 26, 2017 (JP) 2017-207292

(51) Int. Cl. C12M 3/00; C12M 1/34; C12M 1/36; C12M 1/00; G06T 7/00

(52) U.S. Cl. CPC C12M 21/06; C12M 41/46; C12M 41/48; C12M 41/14; C12M 41/12; G06T 2207/20081; C12M 41/26; C12M 41/32; G06T 7/0012; G06T 2207/20076; G06T 2207/30024; C12M 41/34

(57) ABSTRACT

An information processing apparatus according to the present technology includes an acquisition section and a prediction section. The acquisition section acquires a plurality of chronologically captured observation images of a fertilized egg, and culture environment information regarding the fertilized egg. Using the plurality of observation images and the culture environment information, the prediction section predicts a period of time necessary until the fertilized egg reaches a specified form of development, and a quality of the fertilized egg in the form of development.

### TECHNICAL FIELD

The present technology relates to an information processing apparatus, an information processing method, a program, and an observation system that are applicable to assessment of a cell such as a fertilized egg.

### BACKGROUND ART

Conventionally, an observation system is known that assesses the level of quality and the stage of development of a fertilized egg, and presents a result of the assessments. For example, Patent Literature 1 discloses a technology that calculates a specified score used to assess the current stage of development of a cell, using information such as the number of cells, a shape of a cell, a diameter of a cell, and the circularity of a cell.

Further, Patent Literature 2 discloses a technology that assesses the quality of a fertilized egg by measuring a temporal timing of cell division, and a state of synchronous cell division in which a change in form from the 2-cell stage to the 4-cell stage is performed, and then a change in form from the 4-cell stage to the 8-cell stage is performed.

### CITATION LIST

### Patent Literature

Patent Literature 1: Japanese Patent Application Laid-open No. 2015-130806

Patent Literature 2: Japanese Patent Application Laid-open No. 2013-198503

### DISCLOSURE OF INVENTION

### Technical Problem

Patent Literatures 1 and 2 each disclose a technology that assesses the quality and the stage of development of a cell. In recent years, a technology is desired that is capable of predicting, for example a future quality of a cell and presenting a result of the prediction to a user.

In view of the circumstances described above, it is an object of the present technology to provide an information processing apparatus, an information processing method, a program, and an observation system that are capable of presenting a prediction result regarding an observation-target fertilized egg.

### Solution to Problem

In order to achieve the object described above, an information processing apparatus according to an embodiment of the present technology includes an acquisition section and a prediction section.

The acquisition section acquires a plurality of chronologically captured observation images of a fertilized egg, and culture environment information regarding the fertilized egg.

Using the plurality of observation images and the culture environment information, the prediction section predicts a period of time necessary until the fertilized egg reaches a specified form of development, and a quality of the fertilized egg in the form of development.

According to this configuration, a period of time necessary until a fertilized egg reaches a specified form of development, and a quality of the fertilized egg in the form of development are predicted. This enables a user to confirm a future prediction result regarding a fertilized egg of an observation target. Thus, the present technology makes it possible to provide an information processing apparatus that is capable of presenting a prediction result regarding a fertilized egg of an observation target.

The prediction section may include a predictor that is generated using an algorithm in which a plurality of pieces of image data of chronologically captured images of a fertilized egg, and culture environment data of the fertilized egg are training data, and using the plurality of observation images and the culture environment information, the predictor may predict the necessary period of time and the quality of the fertilized egg in the form of development.

Using the plurality of observation images and the culture environment information, the predictor may calculate a probability distribution of the period of time necessary until a fertilized egg reaches the form of development, and a quality score that indicates a quality of the fertilized egg.

The predictor may calculate, as the quality score, a quality of a fertilized egg after an elapse of a necessary period of time, in which the probability of the fertilized egg reaching the form of development after the elapse of the necessary period of time, is highest in the probability distribution.

The acquisition section may further acquire an acquisition request to acquire a fertilized egg cultured for a specified period of time, and

a culture environment controller that controls a culture environment of a fertilized egg according to adjustment parameter information regarding the culture environment may further be included, the adjustment parameter information being generated by the predictor using the plurality of observation images, the culture environment information, and the acquisition request.

This results in a culture environment for culturing a fertilized egg being controlled to comply with a request made by a user.

A determination section that determines whether the quality score for the fertilized egg cultured for the specified period of time is not less than a specified threshold, may further be included.

The culture environment controller may include a recognizer that is generated using an algorithm in which environment setting data is training data, and

when the determination section has determined that the quality score for the fertilized egg cultured for the specified period of time is not less than the specified threshold, the culture environment controller may control the culture environment of the fertilized egg by applying the adjustment parameter information to the recognizer.

This makes it possible to control the growth rate of a fertilized egg while maintaining the quality of the fertilized egg.

According to the adjustment parameter information, the culture environment controller may control at least one of pH of a culture solution used to culture a fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.

The acquisition section may acquire, as the culture environment information, at least one of pH of a culture solution used to culture the fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.

Using the plurality of observation images and the culture environment information, the prediction section may predict a period of time necessary until the fertilized egg reaches early blastocyst, blastocyst, or expanded blastocyst.

In order to achieve the object described above, in an information processing method according to an embodiment of the present technology, a plurality of chronologically captured observation images of a fertilized egg, and culture environment information regarding the fertilized egg are acquired.

Using the plurality of observation images and the culture environment information, a period of time necessary until the fertilized egg reaches a specified form of development, and a quality of the fertilized egg in the form of development are predicted.

Further, in the information processing method, an acquisition request to acquire the fertilized egg cultured for a specified period of time may be acquired;

adjustment parameter information regarding a culture environment of the fertilized egg may be generated using the plurality of observation images, the culture environment information, and the acquisition request; and

the culture environment of the fertilized egg may be controlled according to the adjustment parameter information.

In the predicting the period of time necessary until the fertilized egg reaches the specified form of development, and the quality of the fertilized egg in the form of development, a probability distribution of the period of time necessary until the fertilized egg reaches the form of development, and a quality score that indicates a quality of the fertilized egg may be calculated.

In the information processing method,

further, whether the quality score for the fertilized egg cultured for the specified period of time is not less than a specified threshold may be determined, and

in the controlling the culture environment of the fertilized egg, the culture environment of the fertilized egg may be controlled according to the adjustment parameter information when the quality score for the fertilized egg cultured for the specified period of time has been determined to not be less than the specified threshold.

In order to achieve the object described above, a program according to an embodiment of the present technology causes an information processing apparatus to perform a process including:

acquiring a plurality of chronologically captured observation images of a fertilized egg, and culture environment information regarding the fertilized egg; and

predicting, using the plurality of observation images and the culture environment information, a period of time necessary until the fertilized egg reaches a specified form of development, and a quality of the fertilized egg in the form of development.

In order to achieve the object described above, an observation system according to an embodiment of the present technology includes an observation apparatus, an incubator, a detector, an information processing apparatus, and a display section.

The observation apparatus includes an image-capturing section that chronologically captures images of a fertilized egg, a light source that irradiates light onto the fertilized egg, and a culture vessel that contains the fertilized egg and a culture solution.

The incubator accommodates the observation apparatus.

The detector is capable of detecting a temperature, humidity, and an oxygen concentration in the incubator, pH and an osmotic pressure of the culture solution, concentrations of hormone and a nutrient that are included in the culture solution, a partial pressure of oxygen and a partial pressure of carbon dioxide in the incubator, and illumination intensity of the light source.

The information processing apparatus includes an acquisition section and a prediction section.

The acquisition section acquires a plurality of observation images chronologically captured by the image-capturing section, and a result of the detection performed by the detector, the plurality of observation images being a plurality of observation images of the fertilized egg.

Using the plurality of observation images and the result of the detection, the prediction section predicts a period of time necessary until the fertilized egg reaches a specified form of development, and a quality of the fertilized egg in the form of development.

The display section displays the plurality of observation images and a result of the prediction regarding the fertilized egg.

The observation system may further include an input section that receives an input of an acquisition request to acquire the fertilized egg cultured for a specified period of time,

using the plurality of observation images, the result of the detection, and the acquisition request, the prediction section may further generate adjustment parameter information regarding a culture environment of the fertilized egg, and

the information processing apparatus may further include a culture environment controller that controls, according to the adjustment parameter information, at least one of pH of a culture solution used to culture the fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.

### Advantageous Effects of Invention

As described above, the present technology makes it possible to provide an information processing apparatus, an information processing method, a program, and an observation system that are capable of presenting a prediction result regarding an observation-target fertilized egg. Note that the effect described above is not necessarily limitative, and any effect described in this specification or other effects that could be understood from this specification may be provided in addition to, or instead of the effect described above.

### BRIEF DESCRIPTION OF DRAWINGS

FIG. 1 schematically illustrates an example of a configuration of an observation system according to an embodiment of the present technology.

FIG. 2 schematically illustrates a culture vessel group provided on an observation stage of the observation system, as viewed from the side of a light source.

FIG. 3 schematically illustrates a cross section of a culture vessel according to the embodiment.

FIG. 4 schematically illustrates the culture vessel, as viewed from the side of the light source.

FIG. 5 schematically illustrates an image-capturing area in the culture vessel, as viewed from the side of the light source.

FIG. 6 is a block diagram of the example of the configuration of the observation system.

FIG. 7 is a flowchart of a processing method regarding a fertilized egg that is performed by an information processing apparatus of the embodiment.

FIG. 8 schematically illustrates a state in which an image-capturing section of the observation system captures images of a plurality of fertilized eggs.

FIG. 9 is a conceptual diagram virtually illustrating a first chronological image.

FIG. 10 is a conceptual diagram virtually illustrating a second chronological image.

FIG. 11 is a block diagram simply illustrating a procedure of processing performed by typical narrow AI.

FIG. 12 illustrates a network configuration of an RNN that is a machine learning algorithm.

FIG. 13 illustrates an example of a display state in which a prediction result regarding a fertilized egg is displayed on a display section of the embodiment.

FIG. 14 illustrates an example of a display state in which a prediction result regarding a fertilized egg is displayed on the display section of the embodiment.

FIG. 15 illustrates an example of a display state in which a prediction result regarding a fertilized egg is displayed on the display section of the embodiment.

FIG. 16 illustrates an example of a display state in which a prediction result regarding a fertilized egg is displayed on the display section of the embodiment.

FIG. 17 illustrates a network configuration of an MLP that is a machine learning algorithm.

FIG. 18 illustrates an example of a display state of a prediction result regarding a fertilized egg according to a modification of the present technology.

FIG. 19 illustrates an example of a display state of a prediction result regarding a fertilized egg according to the modification of the present technology.

FIG. 20 schematically illustrates an example of a configuration of an observation system according to another embodiment of the present technology.

FIG. 21 is a block diagram of the example of the configuration of the observation system.

FIG. 22 is a flowchart of a processing method regarding a fertilized egg that is performed by an information processing apparatus of the other embodiment.

### MODE(S) FOR CARRYING OUT THE INVENTION

Embodiments of the present technology will now be described below with reference to the drawings. In the figures, an X-axis, a Y-axis, and a Z-axis that are orthogonal to one another are illustrated as appropriate. Here, an X-axis direction and a Y-axis direction correspond to the horizontal direction, and a Z-axis direction corresponds to the vertical direction. Note that the definitions of the X-axis, the Y-axis, and the Z-axis are the same in all of the figures.

<Configuration of Observation System>

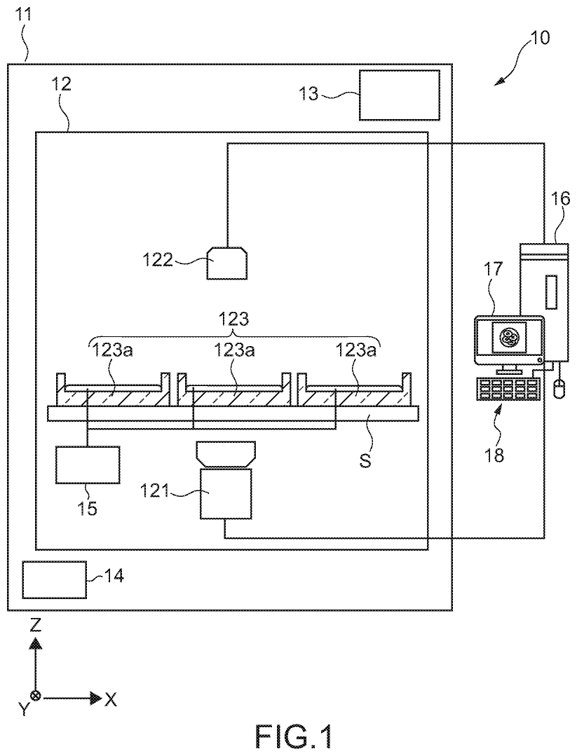

FIG. 1 schematically illustrates an example of a configuration of an observation system 10 according to an embodiment of the present technology. As illustrated in FIG. 1, the observation system 10 includes an incubator 11, an observation apparatus 12, a humidity-temperature-gas controller 13, a detector 14, a culture solution adjusting section 15, an information processing apparatus 16, a display section 17, and an input section 18.

The incubator 11 is a culture apparatus that accommodates the observation apparatus 12, the humidity-temperature-gas controller 13, the detector 14, and the culture solution adjusting section 15, and the incubator 11 has a function that keeps, for example, a temperature and humidity inside the incubator 11 constant. Any gas flows into the incubator 11 of the present embodiment. The type of the gas is not particularly limited, and is, for example, nitrogen, oxygen, or carbon dioxide.

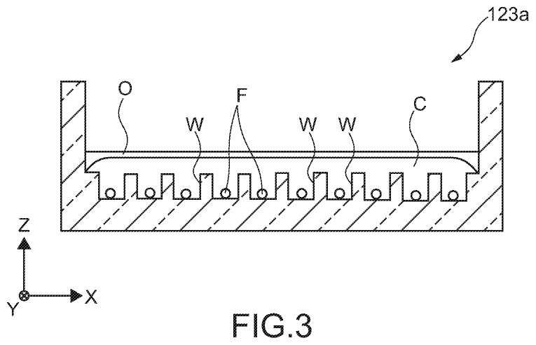

The observation apparatus 12 includes an image-capturing section 121, a light source 122, and a culture vessel group 123. The image-capturing section 121 is capable of chronologically capturing images of a fertilized egg F (refer to FIG. 3) contained in a culture vessel 123a (a dish) to generate the images of the fertilized egg F.

The image-capturing section 121 includes, for example, a lens barrel including a lens group that is movable in an optical-axis direction (the Z-axis direction), a fixed imaging element such as a complementary metal oxide semiconductor (CMOS) or a charge coupled device (CCD) that receives subject light that passes through the lens barrel, and a drive circuit that drives them.

The image-capturing section 121 is movable in directions of three axes that are the X-axis direction, the Y-axis direction, and the Z-axis direction, and captures an image of a fertilized egg F contained in the culture vessel 123a while moving in the horizontal direction (the X-axis direction and the Y-axis direction). Further, the image-capturing section 121 may be capable of capturing not only a still image but also a moving image.

Although the image-capturing section 121 of the present embodiment is typically a visible-light camera, the image-capturing section 121 is not limited to this, and may be, for example, an infrared (IR) camera or a polarization camera.

The light source 122 irradiates light onto the culture vessel 123a when an image of a fertilized egg F contained in the culture vessel 123a is captured using the image-capturing section 121. For example, a light emitting diode (LED) that irradiates light of a specific wavelength is adopted as the light source 122. When the light source 122 is an LED, for example, a red LED is adopted that irradiates light of a wavelength of 640 nm.

The culture vessel group 123 includes a plurality of culture vessels 123a, and is provided on an observation stage S between the image-capturing section 121 and the light source 122. The observation stage S is configured such that light irradiated by the light source 122 can be transmitted through the observation stage S.

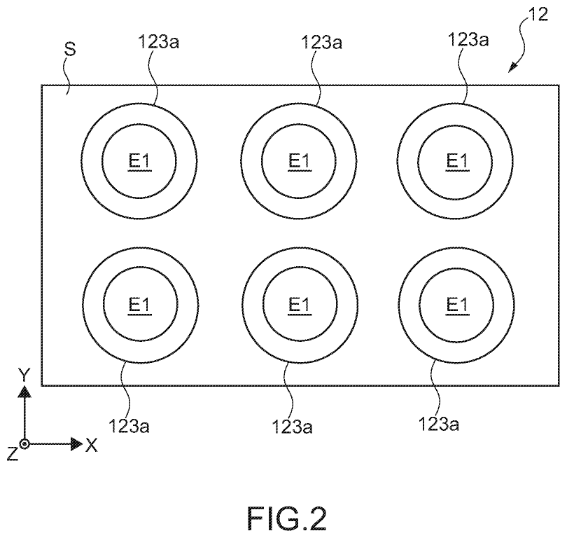

FIG. 2 schematically illustrates the culture vessel group 123 provided on the observation stage S of the observation apparatus 12, as viewed from the side of the light source 122. For example, as illustrated in FIG. 2, six culture vessels 123a are arranged in a matrix on the observation stage S, where three of the six culture vessels 123a are arranged in the X-axis direction and two of the six culture vessels 123a are arranged in the Y-axis direction.

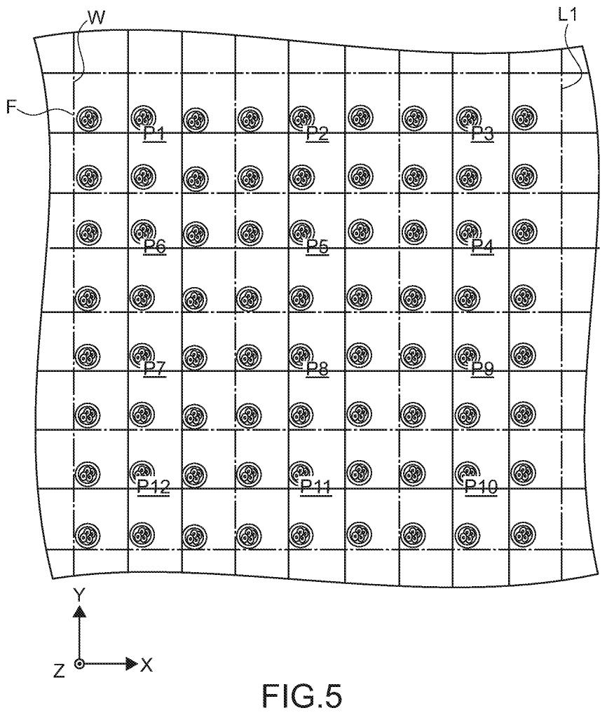

FIG. 3 schematically illustrates a cross section of the culture vessel 123a. As illustrated in FIG. 3, the culture vessel 123a includes a plurality of wells W. The wells W are provided in a matrix (refer to FIG. 5) in the culture vessel 123a, and each well W is capable of containing one fertilized egg F.

In addition to including the well W, the culture vessel 123a contains a culture solution C and oil 0. The oil 0 includes a function that prevents the evaporation of the culture solution C by being coated on the culture solution C.

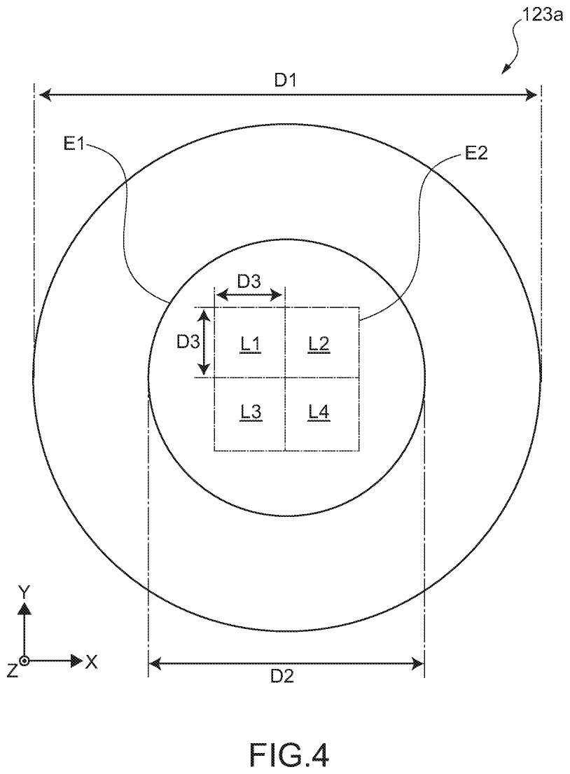

FIG. 4 is a schematic diagram (a plan view) of the culture vessel 123a, as viewed from the side of the light source 122. The culture vessel 123a includes a well region E1 in which a plurality of wells W is formed. A diameter D1 of the culture vessel 123a and a diameter D2 of the well region E1 are not particularly limited, and, for example, the diameter D1 is about 35 mm, and the diameter D2 is about 20 mm.

The well region E1 includes an image-capturing region E2 that is an image-capturing target of the image-capturing section 121. As illustrated in FIG. 2, the image-capturing region E2 is equally divided into four image-capturing areas L1 to L4. A length D3 of a side of each of the image-capturing areas L1 to L4 is, for example, about 5 mm.

FIG. 5 is a schematic enlarged diagram of the image-capturing area L1, as viewed from the side of the light source 122. The image-capturing area L1 includes 72 wells W from among the plurality of wells W provided in the well region E1, and is equally divided into twelve regions for each position (POS) region.

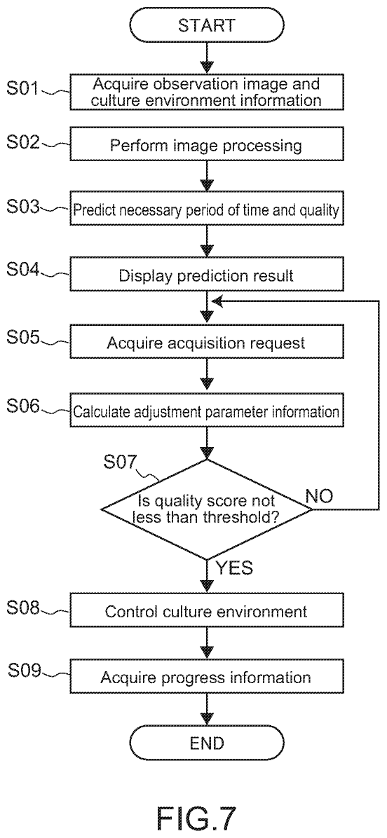

The POS regions P1 to P12 each include six wells W, where three of the six wells W are arranged in the X-axis direction, and two of the six wells W are arranged in the Y-axis direction. In “acquiring observation image and culture environment information” (refer to FIG. 7) described later, the image-capturing section 21 of the present embodiment chronologically captures, for each POS region, images of a fertilized egg F contained in the well W. Note that FIG. 5 is a schematic enlarged diagram of the image-capturing area L1, and the image-capturing areas L2 to L4 each have a configuration similar to the image-capturing area L1.

Although the material of which the culture vessel 123a is made is not particularly limited, the culture vessel 123a is made of, for example, inorganic material such as glass or silicon; or organic material such as polystyrene resin, polyethylene resin, polypropylene resin, ABS resin, nylon, acrylic resin, fluororesin, polycarbonate resin, polyurethane resin, methylpentene resin, phenol resin, melamine resin, epoxy resin, or vinyl chloride resin. The culture vessel 123a is a transparent body through which light irradiated by the light source 122 is transmitted. Alternatively, a portion of the culture vessel 123a may be made of a material from among the materials listed above, or made of metallic material, the portion being a portion other than a portion through which light irradiated by the light source 122 is transmitted.

The humidity-temperature-gas controller 13 controls a temperature and humidity in the incubator 11, and gas introduced into the incubator 11, and creates an environment suitable for the development of a fertilized egg F. For example, the humidity-temperature-gas controller 13 can adjust a partial pressure of oxygen, the oxygen concentration, and a partial pressure of carbon dioxide in the incubator 11.

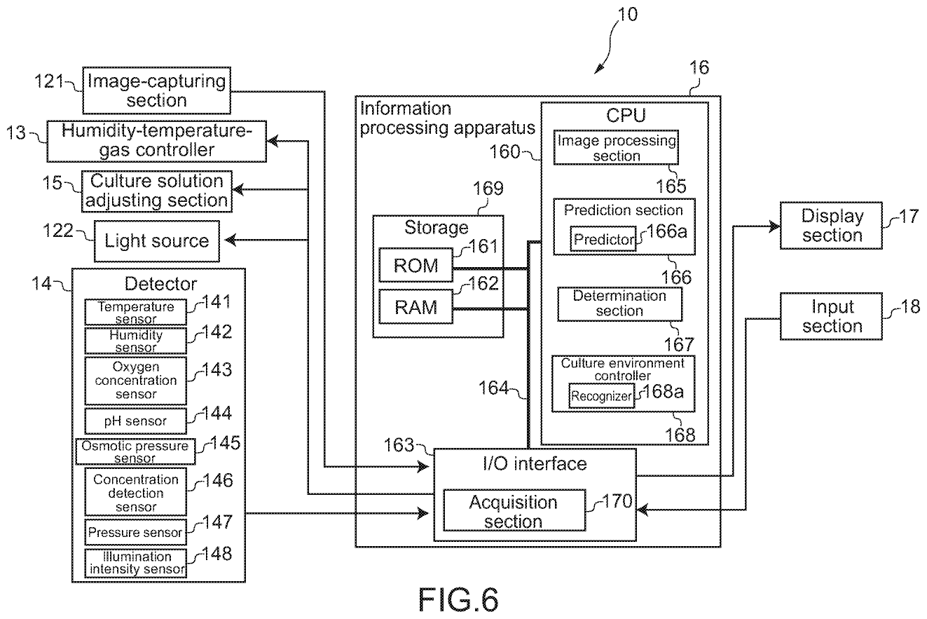

The detector 14 is connected to the information processing apparatus 16 wirelessly or by wire, and includes a temperature sensor 141, a humidity sensor 142, an oxygen concentration sensor 143, a pH sensor 144, an osmotic pressure sensor 145, a concentration detection sensor 146, a pressure sensor 147, and an illumination intensity sensor 148 (refer to FIG. 6).

The temperature sensor 141 detects the temperature in the incubator 11, and outputs a result of the detection to the information processing apparatus 16. For example, a contact device such as a thermocouple, a resistance temperature detector, a thermistor, an IC temperature sensor, or an alcohol thermometer can be adopted as the temperature sensor 141. Alternatively, the temperature sensor 141 may be a contactless device such as a pyroelectric temperature sensor, a thermopile, or a radiation thermometer.

The humidity sensor 142 detects humidity in the incubator 11, and outputs a result of the detection to the information processing apparatus 16. For example, a device of a type such as an impedance-change type, a capacitance-change type, an electromagnetic-wave-absorption type, a heat-transfer-applying type, a wet-and-dry-bulb type, an electromagnetic-wave-absorption type, or a quartz oscillating type may be adopted as the humidity sensor 142, and any type may be used.

The oxygen concentration sensor 143 detects an oxygen concentration in the incubator 11, and outputs a result of the detection to the information processing apparatus 16. For example, a device of a type such as a galvanic type, a polarographic type, a zirconia type, or a fluorescence type can be adopted as the oxygen concentration sensor 143, and any type may be used.

The pH sensor 144 detects pH (hydrogen ion exponent) of a culture solution C used to culture a fertilized egg F, and outputs a result of the detection to the information processing apparatus 16. For example, a device of a type such as a needle type, an implant type, a sleep type, an ultrafine type, a flat type, a sticking type, or a flow-through type can be adopted as the pH sensor 144, and any type may be used.

The osmotic pressure sensor 145 detects an osmotic pressure of a culture solution C on a fertilized egg F, and outputs a result of the detection to the information processing apparatus 16. The concentration detection sensor 146 detects a concentration of a nutrient or hormone that is included in the culture solution C, and outputs a result of the detection to the information processing apparatus 16.

Examples of a nutrient in the culture solution C that can be detected by the concentration detection sensor 146 include amino acid, mineral, inorganic salt, sugar, vitamin, fat, a growth factor, or a mixture thereof. Further, examples of hormone in the culture solution C that can be detected by the concentration detection sensor 146 include auxin such as indoleacetic acid, naphthalene acetic acid, p-chlorophenoxyisobutyric acid, and 2,4-dichlorophenoxyacetic acid; and cytokinin such as kinetin, zeatin, and benzyladenine.

The pressure sensor 147 detects a partial pressure of gas flowing into the incubator 11, and outputs a result of the detection to the information processing apparatus 16. The pressure sensor 147 of the present embodiment typically detects a partial pressure of oxygen and a partial pressure of carbon dioxide in the incubator 11. The type of the pressure sensor 147 is not particularly limited. For example, a device of a type such as a strain-gauge-resistance type, a semiconductor piezoresistive type, a capacitance type, or a silicon resonant type can be adopted, and any type may be used.

The illumination intensity sensor 148 detects the illumination intensity of the light source 122 that irradiates light onto a fertilized egg F, and outputs a result of the detection to the information processing apparatus 16. The type of the illumination intensity sensor 148 is not particularly limited. For example, a device of a type such as a phototransistor type, a photodiode type, or a type obtained by adding an amplifier circuit to a photodiode can be adopted, and any type may be used.

The culture solution adjusting section 15 is connected to a culture solution C injected into the culture vessel 123a, and is capable of adjusting pH and an osmotic pressure of the culture solution C, or a concentration of hormone or a nutrient that is included in the culture solution C.

FIG. 6 is a block diagram of the example of the configuration of the observation system 10. The information processing apparatus 16 includes hardware, such as a central processing unit (CPU) 160, a read only memory (ROM) 161, a random access memory (RAM) 162, an I/O interface 163, and a bus 164, that is necessary for a computer.

The CPU 160 loads, into the RAM 162, a program according to the present technology that is stored in the ROM 161, and executes the program. Accordingly, an operation of each block of the information processing apparatus 16 is controlled. The CPU 160 includes an image processing section 165, a prediction section 166, a determination section 167, and a culture environment controller 168 that will be described later.

A program is installed on the information processing apparatus 16 through, for example, various storage media (internal memory). Alternatively, the installation of a program may be performed through, for example, the Internet. In the present embodiment, for example, a personal computer (PC) is used as the information processing apparatus 16, but any other computers such as a smart device may be used.

The ROM 161 is a memory device that statically stores therein various data and programs that are used in the information processing apparatus 16.

The RAM 162 is used as, for example, a work region for the CPU 160 and a space for temporarily saving historical data. The RAM 162 is a memory device such as a static random access memory (SRAM).

The I/O interface 163 is connected to the CPU 160, a storage 169, the humidity-temperature-gas controller 13, the detector 14, the culture solution adjusting section 15, the display section 17, the input section 18, the image-capturing section 121, and the light source 122, and includes an acquisition section 170. The I/O interface 163 serves as an input/output interface of the information processing apparatus 16.

The bus 164 is a signal transmission path used to perform input and output of various signals between each section of the information processing apparatus 16. The CPU 160, the ROM 161, the RAM 162, and the I/O interface 163 described above are connected to one another through the bus 164.

The image processing section 165 performs specified image processing on a plurality of chronologically captured observation images of a fertilized egg F.

Using a plurality of observation images and culture environment information, the prediction section 166 predicts a period of time necessary until a fertilized egg F reaches a specified form of development, and a quality of the fertilized egg F in the form of development.

The determination section 167 determines whether a quality score that indicates a quality of a fertilized egg F cultured for a specified period of time is not less than a specified threshold, and determines whether to comply with an acquisition request made by a user.

The culture environment controller 168 controls the culture environment of a fertilized egg according to adjustment parameter information that is generated by a predictor 166a using a plurality of observation images, culture environment information, and an acquisition request.

The storage 169 includes, for example, the ROM 161 having stored therein a program executed by the CPU 160, and the RAM 162 used as, for example, a work memory when the CPU 160 performs processing. The storage 169 may further include a nonvolatile memory such as a hard disc drive (HDD) and a flash memory (solid state drive, SSD). This enables the storage 169 to store therein, for example, a plurality of observation images and culture environment information.

The acquisition section 170 acquires a plurality of chronologically captured observation images of a fertilized egg F, and culture environment information regarding the fertilized egg F.

The display section 17 is capable of displaying, for example, a plurality of observation images chronologically captured by the image-capturing section 121, the plurality of observation images being a plurality of observation images of a fertilized egg F. The display section 17 is a display device that uses, for example, liquid crystal or organic electro-luminescence (EL).

The input section 18 is a manipulation device, such as a keyboard and a mouse, that receives an input performed by a user. The input section 18 according to the present embodiment may be, for example, a touch panel integrated with the display section 17.

Note that the functions of the image processing section 165, the prediction section 166, the determination section 167, the culture environment controller 168, the storage 169, and the acquisition section 170 are not limited to those described above, and are described in detail in a description of an information processing method described later.

<Information Processing Method>

FIG. 7 is a flowchart of an information processing method performed by the information processing apparatus 16. The information processing method of the present embodiment is described below with reference to FIG. 7 as appropriate.

[Step S01: Acquisition of Observation Image and Culture Environment Information]

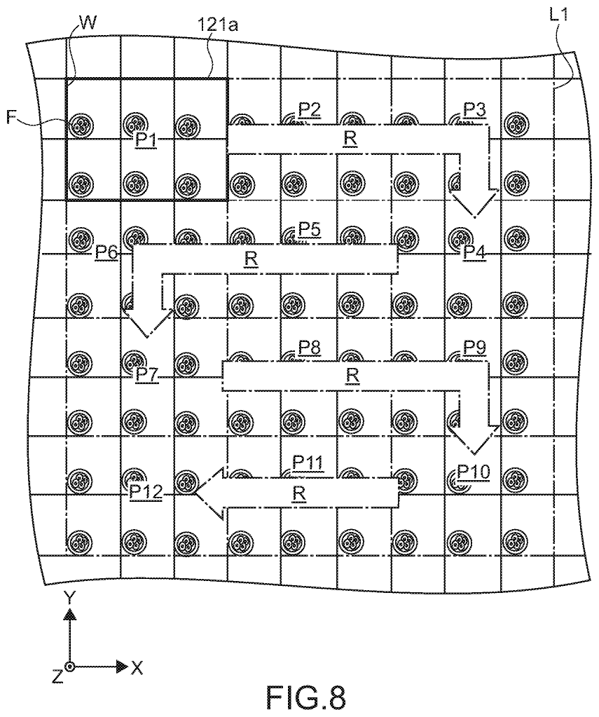

FIG. 8 schematically illustrates a state in which the image-capturing section 121 captures images of a plurality of fertilized eggs F, and illustrates a movement route of the image-capturing section 121.

First, the image-capturing section 121 chronologically captures, for each position (POS) region, images of a plurality of fertilized eggs F each contained in a corresponding one of a plurality of wells W. Here, as illustrated in FIG. 8, a range 121a of a field of view of the image-capturing section 121 moves at intervals of about three seconds from a POS region P1 to a POS region P12 in this order along a movement route R.

Then, this operation is performed with respect to all of the culture vessels 123a provided on the observation stage S, which is repeated a prescribed number of times. Accordingly, images each including six fertilized eggs F (hereinafter referred to as a first chronological image G1) are generated, and the first chronological image G1 is output to the acquisition section 170.

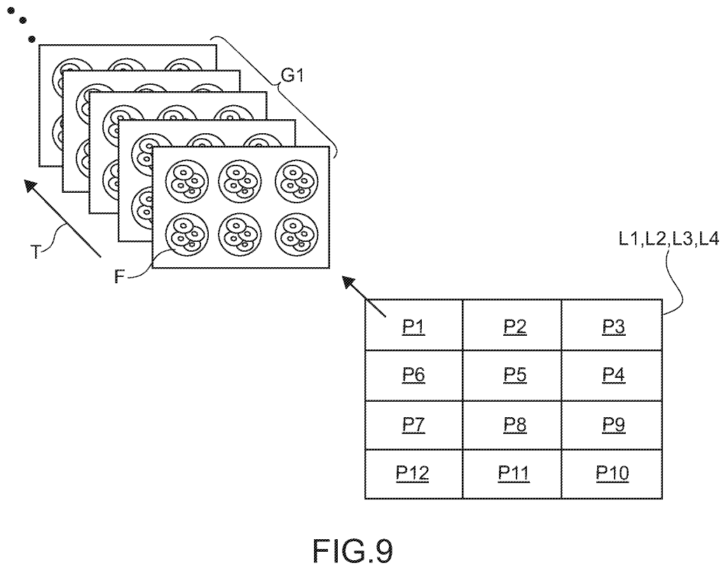

FIG. 9 is a conceptual diagram virtually illustrating the first chronological image G1. Images of the first chronological image G1 of the present embodiment are chronologically generated along a time axis T for each of the POS ranges P1 to P12, as illustrated in FIG. 9. As used herein, an image group illustrated in FIG. 9 is referred to as the first chronological image G1.

The image-capturing interval and the number of images captured of the image-capturing section 121 in the observation system 10 can be set discretionarily. For example, when the image-capturing period of time is one week, the image-capturing interval is 15 minutes, and image-capturing is performed while the focal length is being changed in the depth direction (the Z-axis direction) to obtain nine images in a stack, about 6000 stacked images each including six fertilized eggs F are obtained for one POS region. This makes it possible to acquire a three-dimensional image of a fertilized egg F.

The acquisition section 170 outputs, to the image processing section 165 and the storage 169, the first chronological image G1 output by the image-capturing section 121, and the first chronological image G1 is stored in the storage 169.

In Step S01, in parallel with chronologically capturing images of a plurality of fertilized eggs F for each POS region, a detection result that is a culture environment in the incubator 11 detected by the detector 14 is output to the acquisition section 170 as culture environment information.

The acquisition section 170 of the present embodiment acquires, as culture environment information, at least one of information regarding pH of a culture solution C; information regarding an osmotic pressure of the culture solution C on a fertilized egg F; information regarding concentrations of hormone and a nutrient that are included in the culture solution C; information regarding a temperature, humidity, and an oxygen concentration in the incubator 11; information regarding a partial pressure of oxygen and a partial pressure of carbon dioxide in the incubator 11; or information regarding illumination intensity of the light source 122. The culture environment information of the present embodiment refers to at least one of these pieces of information.

The acquisition section 170 outputs the culture environment information output by the detector 14 to the prediction section 166 and the storage 169, and the culture environment information is stored in the storage 169.

[Step S02: Image Processing]

The image processing section 165 processes (crops), for each fertilized egg, the first chronological image G1 acquired from the acquisition section 170. Accordingly, images each including one fertilized egg F (hereinafter referred to as a second chronological image G2) are generated. Next, the image processing section 165 outputs the second chronological image G2 to the storage 169, and the second chronological image G2 is stored in the storage 169.

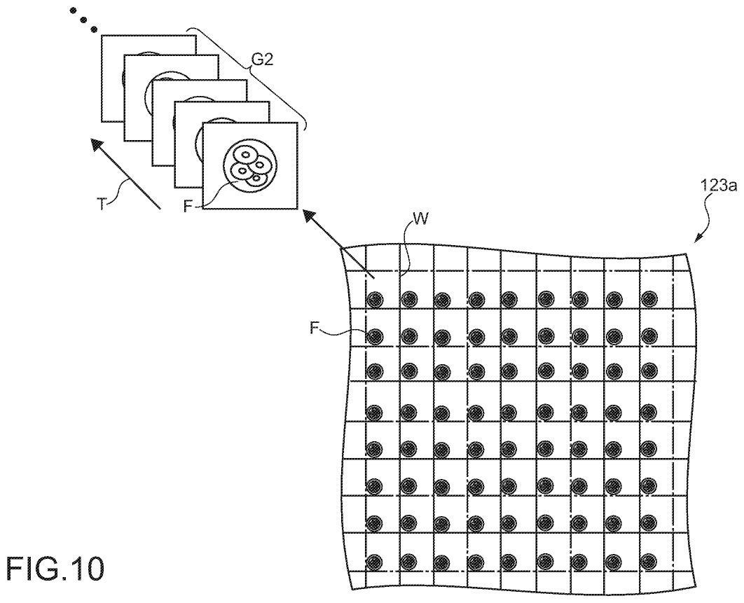

FIG. 10 is a conceptual diagram virtually illustrating the second chronological image G2. Images of the second chronological image G2 of the present embodiment are chronologically generated along the time axis T for a corresponding one of the plurality of wells W, as illustrated in FIG. 10. As used herein, an image group illustrated in FIG. 10 is referred to as the second chronological image G2.

Next, the image processing section 165 performs specified image processing on the second chronological image G2. The second chronological image G2 on which the image processing has been performed by the image processing section 165 is output to the prediction section 166 and the storage 169, and the second chronological image G2 is stored in the storage 169. Application examples of the specified image processing performed by the image processing section 165 are described below.

### Application Example 1

The image processing section 165 performs normalization on respective images included in the second chronological image G2. This results in, for example, removing noise from the second chronological image G2 and thus being able to easily extract the characteristics of the respective images included in the second chronological image G2.

The normalization performed on the second chronological image G2 by the image processing section 165 of the present embodiment is, for example, a normalization process of providing consistency in, for example, color and brightness with respect to the respective images included in the second chronological image G2, or a standardization process, a decorrelating process, or a whitening process.

### Application Example 2

With respect to the second chronological image G2, the image processing section 165 performs, for example, a probability process performed by deep learning analysis, a binarization process, and an overlaying process. This results in, for example, extracting a contour of a fertilized egg F in the second chronological image G2.

### Application Example 3

The image processing section 165 forms a mask region along the shape of a fertilized egg F in each image included in the second chronological image G2. This results in making an analysis region (a recognition region) of a fertilized egg F in the second chronological image G2 clear and thus in being able to accurately recognize the shape of the fertilized egg F. This technology makes it possible to, for example, accurately recognize, for example, the shape of a zona pellucida forming an external configuration of a fertilized egg F, and the shapes of, for example, blastocyst, cell blastomere, and morula in the fertilized egg F.

[Step S03: Prediction of Necessary Period of Time and Quality]

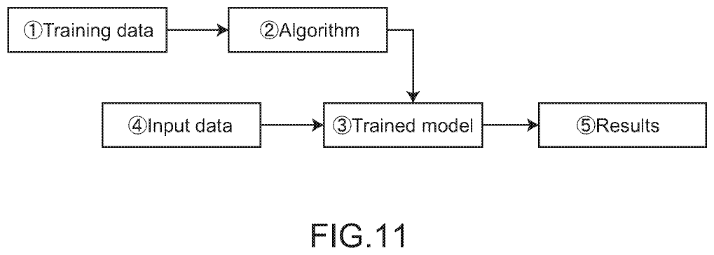

The information processing apparatus 16 of the present embodiment is a computer that uses so-called narrow artificial intelligence (AI) that works on intellectual tasks of a user on his/her behalf. FIG. 11 is a schematic diagram simply illustrating a procedure of processing performed by typical narrow AI.

In a large context, the narrow AI is a system that applies arbitrary input data to a trained model to obtain results, the trained model being built by incorporating training data into an algorithm that serves as a training program. Step S03 is described below with reference to FIG. 11 as appropriate.

The prediction section 166 reads, from the storage 169, culture environment data regarding a fertilized egg that is similar to a fertilized egg F stored in the storage 169 in advance, and a plurality of pieces of image data of chronologically captured images of the fertilized egg. These pieces of information correspond to “Training data” in FIG. 11.

Next, the prediction section 166 builds the predictor 166a by incorporating, into an algorithm set in advance, the training data (the culture environment data and the plurality of pieces of image data) read from the storage 169. Accordingly, the prediction section 166 includes the predictor 166a.

Note that the algorithm described above corresponds to “Algorithm” in FIG. 11, and serves as, for example, a machine learning algorithm. Further, the predictor 166a corresponds to “Trained model” in FIG. 11. Although the predictor 166a of the present embodiment typically includes a single trained model, the configuration is not limited to this, and the predictor 166a may include, for example, a combination of a plurality of trained models.

The type of the machine learning algorithm is not particularly limited, and the machine learning algorithm may be an algorithm that uses neural network such as a recurrent neural network (RNN), a convolutional neural network (CNN), or multilayer perceptron (MLP). Further, the machine learning algorithm may be any algorithm that performs, for example, supervised learning (such as boosting, support vector machine (SVM), and support vector regression (SVR)), unsupervised learning, semi-supervised learning, or reinforcement learning.

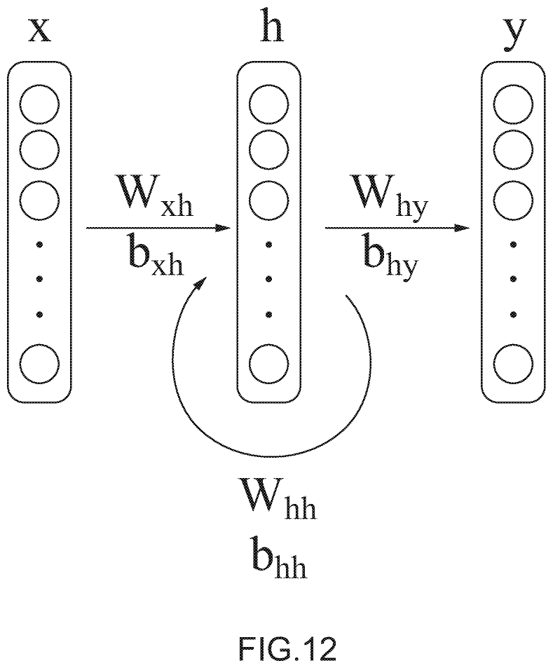

In the present embodiment, an RNN is typically adopted as an algorithm used to build the predictor 166a. FIG. 12 illustrates a network configuration of an RNN.

The RNN is a type of neural network, and has a configuration in which feedback has been added to a hidden layer, as illustrated in FIG. 12. The feedback plays a role in inputting, at a time, a value of a hidden layer of the most previous time, and when pieces of data chronologically related to one another are successively input, the feedback plays a role in extracting pieces of information chronologically related to one another to output a recognition result. Such a function makes it possible to perform recognition using chronological information.

When an input feature amount at a time t is xₜ, and a state of a hidden layer of the most previous time is hₜ₋₁, a function fₜ(x) representing a value of an output layer can be represented using Formula (1) indicated below.

[Formula 1]

fₜ(x)=b_{hy}+W_{hy}(s(bₓₕ+Wₓₕxₜ+bₕₕ+Wₕₕhₜ₋₁)) (Formula 1)

Here, bₓₕ and b_{hy} each represent a bias, Wₓₕ and W_{hy} each represent a weight matrix, where an index xh represents a connection between input and a hidden layer, and an index by represents a connection between the hidden layer and an output layer. S represents an activation function, and, for example, a logistic sigmoid function can be used as the activation function. The logistic sigmoid function is represented using Formula (2) indicated below.

$$\begin{matrix}[ Formula 2 ] & \\ s ( a ) = \frac{1}{1 + {e}^{- a}} & ( Formula 2 )\end{matrix}$$

N data sets of a feature amount x of training data and a prediction label y are represented by (xₙ, yₙ). When parameters of a bias and a weight matrix of the RNN are collectively represented by w, training of a network to estimate a prediction label can be formulated as a problem such as Formula (3) indicated below, the problem being a problem for obtaining a parameter w that makes a value in a formula smallest, such that an output value of Formula (1) is a value as close as possible to the prediction label in training data.

$$\begin{matrix}[ Formula 3 ] & \\ E ( w ) = {{\sum }_{n = 1}}^{N} {‖ {y}_{n} - f ( {x}_{n} ) ‖}_{2}^{2} & ( Formula 3 ) \\ where {‖ \cdot ‖}_{2}^{2} represents L 2 norm . & \end{matrix}$$

In general, Formula (3) is called the Euclidean (L2) loss. For example, this w can be obtained by performing a method, such as stochastic gradient descent, with respect to a training data set. Using a network of RNN obtained by this method, the prediction label y can be calculated from the feature amount x of training data. In other words, it is possible to build the predictor 166a.

Next, by applying the predictor 166a built as described above to the second chronological image G2 and culture environment information regarding a fertilized egg F, the second chronological image G2 being output by the image processing section 165, the culture environment information being associated with the second chronological image G2, the prediction section 166 predicts a period of time necessary until the fertilized egg F reaches a specified form of development, and a quality of the fertilized egg F in this form of development. Then, the prediction section 166 outputs a result of the prediction to the display section 17 or a terminal device 19 described later, and to the storage 169, and the result of the prediction is stored in the storage 169.

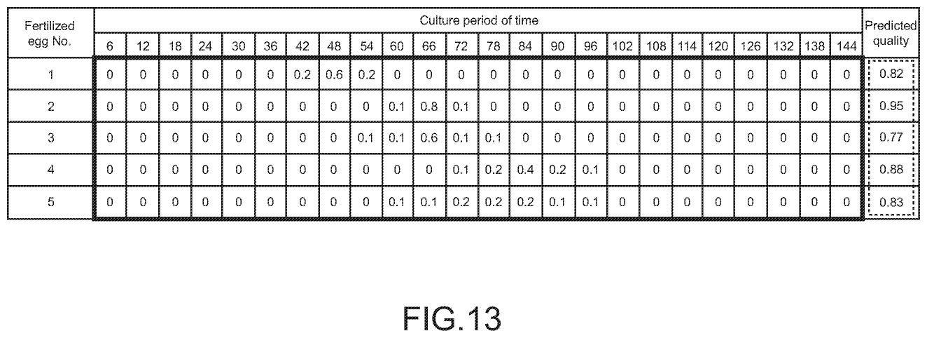

Specifically, by applying the predictor 166a to the second chronological image G2 and culture environment information, the prediction section 166 calculates a probability distribution and a quality score as a prediction result, the probability distribution being a probability distribution of a period of time necessary until a fertilized egg F reaches a specified form of development, the quality score indicating a quality of the fertilized egg F (refer to FIGS. 13 to 15). For example, the quality score is calculated using, for example, a time of a first cleavage, the number of cells upon the first cleavage, and the cellular symmetry or the cellular fragmentation with regard to the fertilized egg F.

Note that, as used herein, the “specified form of development” is not particularly limited as long as it is a form of development to which a fertilized egg F is changed in the process of being cultured, and typically refers to early blastocyst, blastocyst, or expanded blastocyst. Further, the second chronological image G2 and culture environment information associated with the second chronological image G2 correspond to “Input data” in FIG. 11, and the prediction result refers to “Results” in FIG. 11.

[Step S04: Display of Prediction Result]

The display section 17 displays the prediction result output by the prediction section 166. Accordingly, the probability distribution and the quality score described above are presented to a user through the display section 17. Several display examples of the display section 17 are described below.

### Display Example 1

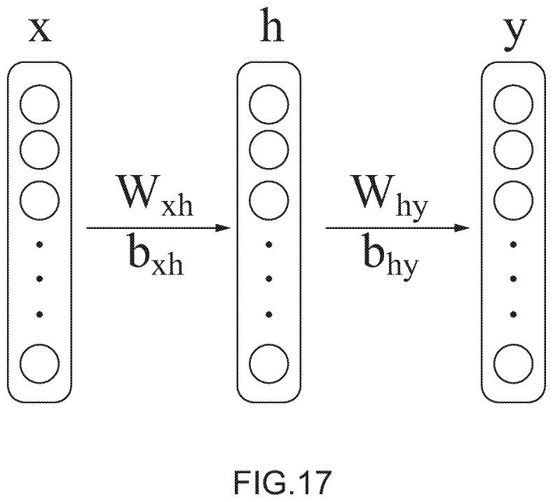

FIG. 13 illustrates an example of a display state in which a prediction result regarding a fertilized egg F is displayed on the display section 17. Here, “Fertilized egg No.” illustrated in FIG. 17 is a number assigned to a fertilized egg F contained in a corresponding one of a plurality of wells W, and is an identification number that identifies each of the fertilized eggs F. Note that, in FIG. 13, numbers 1 to 5 are displayed on the display section 17 as examples of the fertilized egg number, but the fertilized egg number is not limited to this.

Further, “Culture period of time” illustrated in FIG. 13 is a culture period of time necessary until a fertilized egg F reaches a specified form of development, regardless of its time unit such as second, minute, hour, or day. Furthermore, a value corresponding to “Fertilized egg No.” and “Culture period of time” (a value boxed in using bold solid lines in FIG. 13) is a value of the probability that a fertilized egg F will be changed to a specified form of development.

In addition, a value of “Predicted quality” illustrated in FIG. 13 (a value boxed in using dotted lines in FIG. 13) is a quality score that indicates a future quality when a fertilized egg F reaches a specified form of development.

Here, the quality score of the present embodiment is a value that indicates a quality of a fertilized egg F after the elapse of a period of time necessary until the fertilized egg F reaches a specified form of development, in which the probability of the fertilized egg F reaching the specified form of development after the elapse of the necessary period of time, is highest in a probability distribution of the necessary period of time. For example, in the case of an example of a fertilized egg F of fertilized egg No. 1 in FIG. 13, a quality score of “0.82” is a score that indicates a quality of the fertilized egg F after the elapse of a necessary period of time (48) corresponding to a probability value of “0.6”.

### Display Example 2

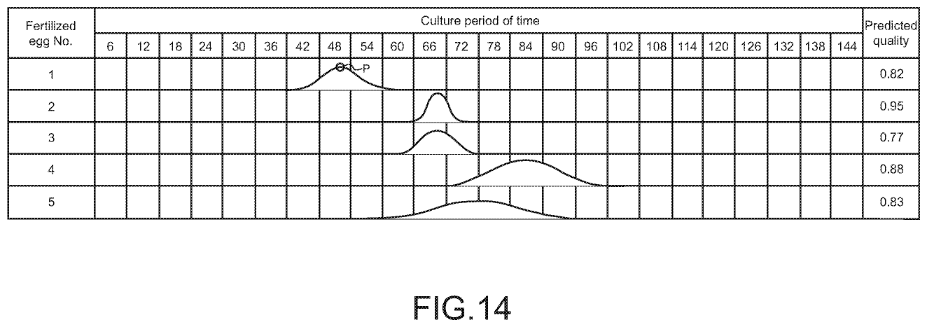

FIG. 14 illustrates another example of a display state in which a prediction result regarding a fertilized egg F is displayed on the display section 17. As illustrated in FIG. 14, in Display Example 2, the probability distribution of a period of time necessary until a fertilized egg F reaches a specified form of development is displayed in the form of a probability graph.

Here, in the case of an example of a fertilized egg F of a fertilized egg No. 1 in FIG. 14, a quality score of “0.82” is a score that indicates a quality of the fertilized egg F after the elapse of a necessary period of time corresponding to a peak P (a largest value) in a probability graph.

[Step S05: Acquisition of Acquisition Request]

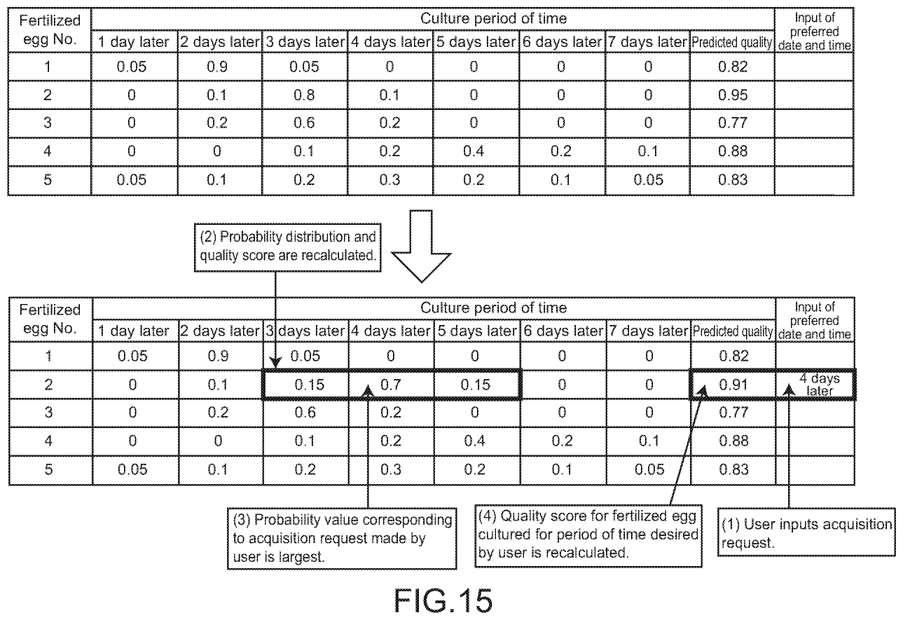

FIG. 15 illustrates an example of a display state of a prediction result regarding a fertilized egg F, and illustrates an example of recalculating a probability distribution and a quality score. Step S05 is described below with reference to FIG. 15.

First, A user who has viewed and assessed a prediction result regarding a fertilized egg F that is displayed on the display section 17 inputs (1), to the input section 18 or the terminal device 19, an acquisition request to acquire a fertilized egg F cultured for a specified period of time. The input section 18 or the terminal device 19 to which the acquisition request has been input by the user outputs the acquisition result to the acquisition section 170.

The acquisition section 170 that has acquired the acquisition request from the input section 18 or the terminal device 19 outputs the acquisition request to the prediction section 166 and the storage 169, and the acquisition request is stored in the storage 169. Accordingly, the period of time necessary until the fertilized egg F reaches a specified form of development, and the quality of the fertilized egg F after the elapse of the specified period of time are repredicted.

Specifically, the prediction section 166 recalculates (2) the probability distribution and the quality score calculated in Step S. Then, the prediction section 166 outputs this prediction result to the determination section 167 and the storage 169, and the prediction result is stored in the storage 169.

Here, the prediction section 166 recalculates (3) the probability distribution such that the probability value regarding a fertilized egg F cultured for a period of time desired by the user is largest, and then the quality score for the fertilized egg F is recalculated (4).

[Step S06: Calculation of Adjustment Parameter Information]

Next, by applying the predictor 166a built in Step S03 described above to the second chronological image G2, culture environment information regarding a fertilized egg F, and the acquisition request, the second chronological image G2 being read from the storage 169, the culture environment information being associated with the second chronological image G2, the prediction section 166 calculates adjustment parameter information regarding the culture environment of the fertilized egg F. Then, the prediction section 166 outputs the calculated adjustment parameter information to the culture environment controller 168 and the storage 169, and this adjustment parameter information is stored in the storage 169.

The prediction section 166 of the present embodiment calculates, as adjustment parameter information, at least one of pH of a culture solution C, an osmotic pressure of the culture solution C, a concentration of hormone included in the culture solution C, a concentration of a nutrient included in the culture solution C, a temperature in the incubator 11, humidity in the incubator 11, an oxygen concentration in the incubator 11, a partial pressure of oxygen in the incubator 11, a partial pressure of carbon dioxide in the incubator 11, or illumination intensity of the light source 122.

[Step S07: Determination of Quality Score]

In Step S07, the determination section 167 determines whether the quality score recalculated in Step S05 described above is not less than a specified threshold. Note that the threshold may be determined discretionarily according to the specifications or the use of the observation system 10.

(NO in Step S07: When the Quality Score is Less than a Specified Threshold)

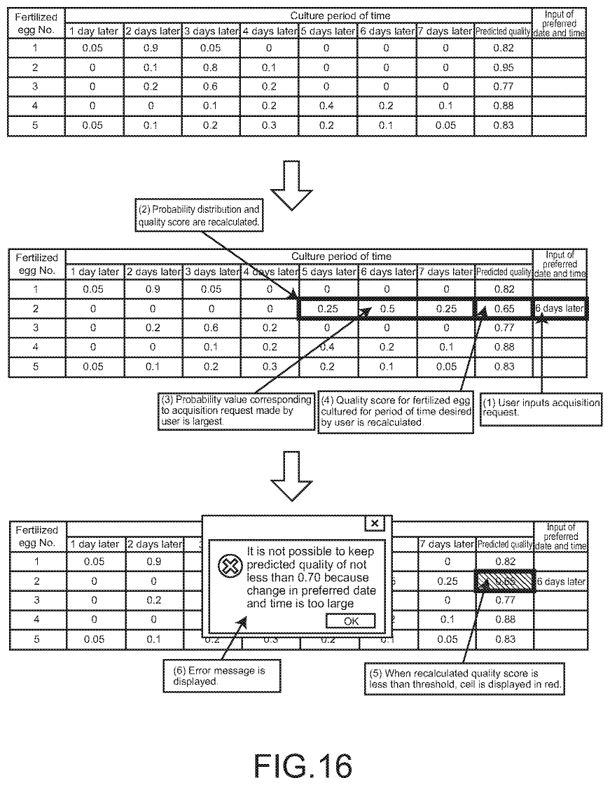

FIG. 16 illustrates an example of a display state of a prediction result regarding a fertilized egg F, and illustrates an example of recalculating a probability distribution and a quality score.

When the quality score recalculated by performing the processes ((1) to (4)) described in Step S05 described above is less than a specified threshold, a cell indicating the quality score is displayed in, for example, red and the acquisition request is refused (5). Then, an error message or the like is displayed on the display section 17 or the terminal device 19, and the user is urged to input (6) an acquisition request again.

(YES in Step S07: When the Quality Score is not Less than the Specified Threshold)

When the quality score recalculated according to the acquisition request made by the user is not less than the specified threshold (YES in S07), the culture environment controller 168 reads, from the storage 169, adjustment parameter information stored in the storage 169 in Steps S05 and S06 described above.

[Step S08: Culture Environment Control]

In Step S08, the culture environment of the fertilized egg F is controlled according to the feature amount of the fertilized egg F and the acquisition request input by the user.

First, the culture environment controller 168 reads, from the storage 169, environment setting data regarding a fertilized egg F similar to the fertilized egg F stored in the storage 169 in advance. This data corresponds to “Training data” in FIG. 11.

The environment setting data refers to various parameters (such as pH of a culture solution; an osmotic pressure of the culture solution; concentrations of hormone and a nutrient that are included in the culture solution; a temperature, humidity, and an oxygen concentration in an incubator; a partial pressure of oxygen and a partial pressure of carbon dioxide in the incubator; and illumination intensity of a light source that irradiates light onto a fertilized egg) and information associated with the various parameters, the various parameters being obtained by culturing a fertilized egg in various culture environments and contributing toward development of a fertilized egg, the information associated with the parameters being information regarding, for example, the quality and the growth rate of a fertilized egg.

Next, the culture environment controller 168 builds a recognizer 168a by incorporating, into an algorithm set in advance, the training data (the environment setting data) read from the storage 169. Accordingly, the culture environment controller 168 includes the recognizer 168a.

Note that the algorithm described above corresponds to “Algorithm” in FIG. 11, and serves as, for example, a machine learning algorithm. Further, the recognizer 168a corresponds to “Trained model” in FIG. 11. Although the recognizer 168a typically includes a single trained model, the configuration is not limited to this, and the recognizer 168a may include, for example, a combination of a plurality of trained models.

In the present embodiment, an MLP is typically adopted as an algorithm used to build the recognizer 168a. FIG. 17 illustrates a network configuration of an MLP.

The MLP is a type of neural network. FIG. 17 illustrates a structure of a 2-layer MLP including a hidden layer. In this case, when an input feature amount is x, a function f(x) representing a value of an output layer can be represented using Formula (4) indicated below.

[Formula 4]

f(x)=b_{hy}+W_{hy}(s(bₓₕ+Wₓₕx)) (Formula 4)

Here, bₓₕ and b_{hy} each represent a bias, Wₓₕ and W_{hy} each represent a weight matrix, where an index xh represents a connection between input and a hidden layer, and an index by represents a connection between the hidden layer and an output layer. S represents an activation function, and, for example, the logistic sigmoid function of Formula (2) can be used as the activation function.

N data sets of a feature amount x of training data and a prediction label y are represented by (xₙ, yₙ). When parameters of a bias and a weight matrix of the MLP are collectively represented by w, training of a network to estimate a prediction label can be formulated as a problem such as Formula (3), the problem being a problem for obtaining a parameter w that makes a value in a formula smallest, such that an output value of Formula (4) is a value as close as possible to the prediction label in training data. Using a network of MLP obtained by the method described above, the prediction label y can be calculated from the feature amount x of training data. In other words, it is possible to build the recognizer 168a.

Next, various setting values are chronologically calculated by the culture environment controller 168 applying the recognizer 168a built as described above to the adjustment parameter information read from the storage 169 in Step S07 described above. Then, the culture environment controller 168 outputs the calculated various setting values to the humidity-temperature-gas controller 13, the culture solution adjusting section 15, the light source 122, and the storage 169, and the various setting values are stored in the storage 169. Accordingly, at least one of the humidity-temperature-gas controller 13, the culture solution adjusting section 15, or the light source 122 is controlled according to the calculated various setting values.

Specifically, according to the adjustment parameter information, the culture environment controller 168 controls at least one of pH of a culture solution C, an osmotic pressure of the culture solution C, a concentration of hormone included in the culture solution C, a concentration of a nutrient included in the culture solution C, a temperature in the incubator 11, humidity in the incubator 11, an oxygen concentration in the incubator 11, a partial pressure of oxygen in the incubator 11, a partial pressure of carbon dioxide in the incubator 11, or illumination intensity of the light source 122.

Accordingly, at least one of the humidity-temperature-gas controller 13, the culture solution adjusting section 15, or the light source 122 is controlled according to the acquisition request input to the input section 18 or the terminal device 19 by the user in Step S05 described above, and the culture environment in the incubator 11 is controlled to comply with the acquisition request made by the user.

Note that the feature amount and the acquisition request described above correspond to “Input data” in FIG. 11, and the various setting values correspond to “Results” in FIG. 11.

[Step S09: Acquisition of Progress Information]

In Step S09, progress information regarding a fertilized egg F acquired by the user, and culture environment information upon acquiring the fertilized egg F are fed back to the information processing apparatus 16, in order to improve the accuracy in analysis.

The user acquires progress information regarding a fertilized egg F acquired by the culture environment in the incubator 11 being controlled according to the acquisition request made by the user himself/herself. The progress information is information regarding, for example, the quality and the form of development of a fertilized egg F, the progress information being obtained by the user, for example, after the culture environment has been controlled.

Next, the user inputs the progress information acquired as described above to the input section 18 or the terminal device 19. Accordingly, the progress information is output from the input section 18 or the terminal device 19 to the acquisition section 170. Then, the acquisition section 170 that has acquired the progress information outputs the progress information to the prediction section 166.

Next, the prediction section 166 that has acquired the progress information from the acquisition section 170 reads, from the storage 169, the second chronological image G2 and the culture environment information regarding a fertilized egg F, the second chronological image G2 and the culture environment information being stored in the storage 169 and being associated with the acquired progress information. Next, the predictor 166a is rebuilt by the prediction section 166 incorporating, into an algorithm set in advance and as training data, the progress information, as well as the second chronological image G2 and the culture environment information that have been read from the storage 169. This results in updating the predictor 166a, and thus being able to perform analysis taking into consideration not only the second chronological image G2 and the culture environment information regarding a fertilized egg F, but also the progress information. This results in improving the accuracy in analysis performed by the prediction section 166.

<Effects>

In the observation system 10 of the present embodiment, a period of time necessary until a fertilized egg F reaches a specified form of development, and a prediction result regarding the quality of the fertilized egg F when the fertilized egg F reaches the specified form of development are presented to a user through the display section 17.

This enables the user to control the quality of a fertilized egg F, and thus to design a plan regarding, for example, implantation and development of the fertilized egg F in the form of blastocyst or in the form subsequent to blastocyst. In particular, the present embodiment makes it possible to design a plan in detail since a prediction result obtained by more accurate analysis being performed using narrow AI, is presented to a user.

Further, in the information processing apparatus 16 of the present embodiment, the culture environment is controlled in compliance with an acquisition request made by a user as long as the quality score recalculated according to the acquisition request made by the user is not less than a specified threshold.

This makes it possible to control the growth rate of a fertilized egg F while maintaining the quality of the fertilized egg F. This enables a cow-calf producer and a fattening farmer to arrange a schedule of implantation and development of a fertilized egg F, and thus makes it possible to enhance the quality of development and to reduce costs. For example, it is possible to develop a high-quality beef at low costs.

### Other Embodiments

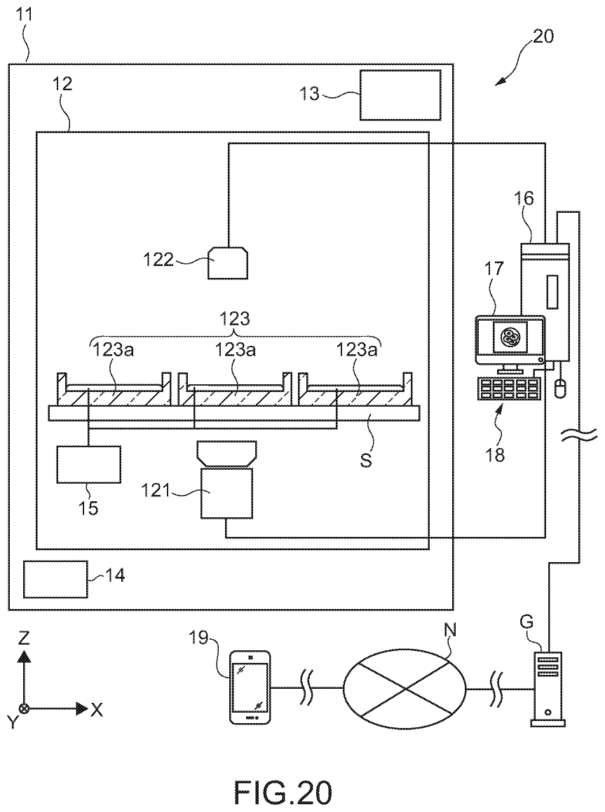

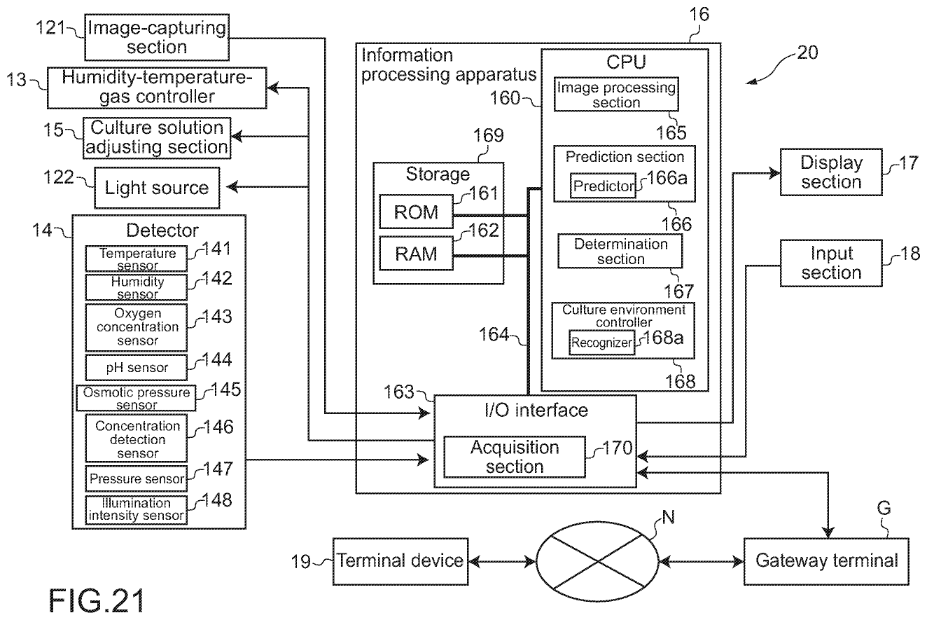

FIG. 20 schematically illustrates an example of a configuration of an observation system 20 according to another embodiment of the present technology, and FIG. 21 is a block diagram of the example of the configuration of the observation system 20. A component similar to that of the embodiment described above is denoted below by the same reference symbol, and a detailed description thereof is omitted.

[Configuration of Observation System]

In the observation system 20 according to another embodiment, the information processing apparatus 16 is connected to a network N through a gateway terminal G, and the network N is connected to the terminal device 19. In other words, the observation system 20 is different from the observation system of the embodiment described above in that the information processing apparatus 16 is connected to the terminal device 19 through the network N.

Note that the observation system 20 is not limited to having the configuration illustrated in FIG. 20, and, for example, the observation system 20 may include a plurality of terminal devices 19 each connected to the network N through the gateway terminal G. Further, the gateway terminal G may be omitted as necessary.

The terminal device 19 is handled by a user. The terminal device 19 displays information acquired from the information processing apparatus 16 through the network N. Specifically, the terminal device 19 acquires a result of sensing in the incubator 11 through the network N, and displays the sensing result on a web browser.

Although the terminal device 19 is typically a smart device, a tablet terminal, or the like, the terminal device 19 is not limited to this, and may be any other computer such as a laptop PC or a desktop PC.

[Information Processing Method]

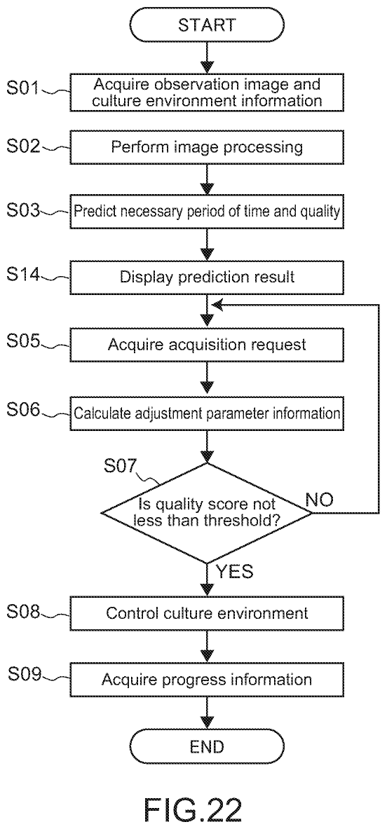

FIG. 22 is a flowchart of an information processing method performed by the information processing apparatus 16. The information processing method of the other embodiment is described below with reference to FIG. 22 as appropriate. Note that descriptions of Steps similar to those of the embodiment described above are omitted.

(Step S14: Display of Prediction Result)

The terminal device 19 displays the prediction result output by the prediction section 166. Accordingly, the probability distribution and the quality score are presented to a user through the terminal device 19, as in the case of the display examples of the embodiment described above (refer to FIGS. 13 and 14).

[Effects]

In the observation system 20 according to the other embodiment of the present technology, the information processing apparatus 16 connected to the detector 14 is connected to the terminal device 19 through the network N. This enables a user to confirm a result of sensing in the incubator in any location and to control the quality and the growth rate of a fertilized egg F according to the result. In other words, a user can remotely control the quality of a fertilized egg F using the terminal device 19. Thus, a user does not necessarily have to stay near the incubator 11, and thus it becomes more convenient in controlling the quality and the growth of a fertilized egg F.

<Modification>

Although the embodiments of the present technology have been described above, of course the present technology is not limited to the embodiments described above and various modifications may be made thereto.

For example, in the observation system 10, a process of capturing images of a fertilized egg F for each arbitrary period of time such as for each specified interval of, for example, 15 minutes or one day, or successively, is repeatedly performed, and a prediction result regarding the fertilized egg F is obtained using images acquired by this process being performed. However, the present technology is not limited to this. In the observation system 10 according to the present embodiment, an image may be acquired in real time as necessary, and a prediction result regarding a fertilized egg F may be displayed as necessary on the display section 17 or the terminal device 19.

Further, in the observation system 10 of the embodiments described above, the culture environment of a fertilized egg F is adjusted by adjusting various parameters (such as pH of a culture solution C; an osmotic pressure of the culture solution C; concentrations of hormone and a nutrient that are included in the culture solution C; a temperature, humidity, and an oxygen concentration in the incubator 11; a partial pressure of oxygen and a partial pressure of carbon dioxide in the incubator 11; and illumination intensity of the light source 122) that contribute toward development of the fertilized egg F. However, the present technology is not limited to this.

For example, in the present technology, the culture environment in the incubator 11 may be controlled by adjusting a culture temperature, culture humidity, or a composition of gas introduced into the incubator 11 according to the quality of a fertilized egg F.

Alternatively, the growth rate of a fertilized egg F may be controlled by using two types of culture solutions (a culture solution for a fertilized egg at an early stage and a culture solution for a fertilized egg at a latter stage) or adjusting pH and an osmotic pressure of a culture solution C, depending on the state of the quality or the form of development of the fertilized egg F.

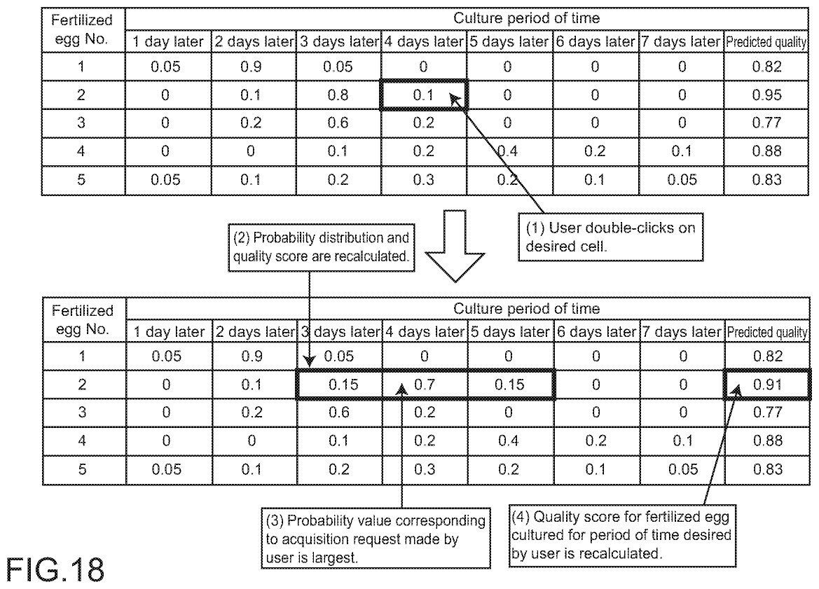

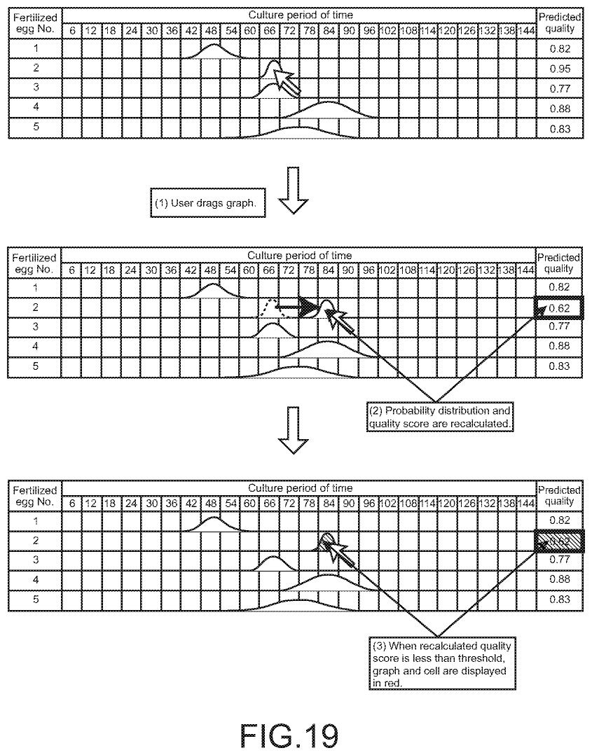

FIGS. 18 and 19 illustrate examples of a display state of a prediction result regarding a fertilized egg according to a modification of the present technology, and illustrate examples of the process of recalculating a probability distribution and a quality score. In the embodiments described above, the quality of a fertilized egg F is repredicted by an acquisition request being input to a cell on a user interface on which a prediction result regarding a fertilized egg F is displayed, but the present technology is not limited to this.

For example, in the present embodiment, a period of time necessary until a fertilized egg F reaches a specified form of development, and a quality of the fertilized egg F after the elapse of the specified period of time may be repredicted by, for example, a user double-clicking on a desired cell, as illustrated in FIG. 18.

Alternatively, as illustrated in FIG. 19, a probability distribution (a probability graph) of a period of time necessary until a fertilized egg F reaches a specified form of development, and a quality score that indicates a quality of the fertilized egg F may be recalculated according to a changed setting by a user dragging the probability graph. In this case, when the recalculated quality score is less than a threshold, the probability graph dragged by the user and the cell indicating the quality score are displayed in, for example, red, and the acquisition request is refused, as illustrated in FIG. 19.

Further, a fertilized egg F observed using the observation system 10 according to the present technology is typically derived from a cow. However, the fertilized egg F is not limited to this, and may be collected from, for example, a mouse, pig, dog, cat, or human.

In addition, as used herein, the “fertilized egg” at least conceptually includes a single cell and a set of cells. Further, the single cell or the set of cells is related to a cell, including to oocyte, egg/ovum, fertile ovum/zygote, blastocyst, and embryo, that is observed in a stage or a plurality of stages in embryonic development.

Further, the present technology is also applicable to any cell such as unfertilized egg cell (ovum) of a living being in the field of, for example, animal husbandry; stem cell in the fields of, for example, regenerative medicine, pathobiology, and gene editing; and immune cell, cancer cell, and the like that are biological samples taken from a living body.

Note that the present technology may also take the following configurations.

(1) An information processing apparatus including:

an acquisition section that acquires a plurality of chronologically captured observation images of a fertilized egg, and culture environment information regarding the fertilized egg; and

a prediction section that predicts, using the plurality of observation images and the culture environment information, a period of time necessary until the fertilized egg reaches a specified form of development, and a quality of the fertilized egg in the form of development.

(2) The information processing apparatus according to (1), in which

the prediction section includes a predictor that is generated using an algorithm in which a plurality of pieces of image data of chronologically captured images of a fertilized egg, and culture environment data of the fertilized egg are training data, and

using the plurality of observation images and the culture environment information, the predictor predicts the necessary period of time and the quality of the fertilized egg in the form of development.

(3) The information processing apparatus (2), in which

using the plurality of observation images and the culture environment information, the predictor calculates a probability distribution of the period of time necessary until a fertilized egg reaches the form of development, and a quality score that indicates a quality of the fertilized egg.

(4) The information processing apparatus according to (3), in which

the predictor calculates, as the quality score, a quality of a fertilized egg after an elapse of a necessary period of time, in which the probability of the fertilized egg reaching the form of development after the elapse of the necessary period of time, is highest in the probability distribution.

(5) The information processing apparatus according to (3) or (4), in which

the acquisition section further acquires an acquisition request to acquire a fertilized egg cultured for a specified period of time, and

the information processing apparatus further includes a culture environment controller that controls a culture environment of a fertilized egg according to adjustment parameter information regarding the culture environment, the adjustment parameter information being generated by the predictor using the plurality of observation images, the culture environment information, and the acquisition request.

(6) The information processing apparatus according to (5), further including a determination section that determines whether the quality score for the fertilized egg cultured for the specified period of time is not less than a specified threshold.
(7) The information processing apparatus according to (6), in which

the culture environment controller includes a recognizer that is generated using an algorithm in which environment setting data is training data, and

when the determination section has determined that the quality score for the fertilized egg cultured for the specified period of time is not less than the specified threshold, the culture environment controller controls the culture environment of the fertilized egg by applying the adjustment parameter information to the recognizer.

(8) The information processing apparatus according to any one of (5) to (7), in which

according to the adjustment parameter information, the culture environment controller controls at least one of pH of a culture solution used to culture a fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.

(9) The information processing apparatus according to any one of (1) to (8), in which

the acquisition section acquires, as the culture environment information, at least one of pH of a culture solution used to culture the fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.

(10) The information processing apparatus according to any one of (1) to (9), in which

using the plurality of observation images and the culture environment information, the prediction section predicts a period of time necessary until the fertilized egg reaches early blastocyst, blastocyst, or expanded blastocyst.

(11) An information processing method including:

acquiring a plurality of chronologically captured observation images of a fertilized egg, and culture environment information regarding the fertilized egg; and

predicting, using the plurality of observation images and the culture environment information, a period of time necessary until the fertilized egg reaches a specified form of development, and a quality of the fertilized egg in the form of development.

(12) The information processing method according to (11), further including:

acquiring an acquisition request to acquire the fertilized egg cultured for a specified period of time;

generating, using the plurality of observation images, the culture environment information, and the acquisition request, adjustment parameter information regarding a culture environment of the fertilized egg; and

controlling the culture environment of the fertilized egg according to the adjustment parameter information.

(13) The information processing method according to (12), in which

further, the predicting the period of time necessary until the fertilized egg reaches the specified form of development, and the quality of the fertilized egg in the form of development includes calculating a probability distribution of the period of time necessary until the fertilized egg reaches the form of development, and a quality score that indicates a quality of the fertilized egg.

(14) The information processing method according to (13), further including determining whether the quality score for the fertilized egg cultured for the specified period of time is not less than a specified threshold, in which

the controlling the culture environment of the fertilized egg includes controlling the culture environment of the fertilized egg according to the adjustment parameter information when the quality score for the fertilized egg cultured for the specified period of time has been determined to not be less than the specified threshold.

(15) A program that causes an information processing apparatus to perform a process including:

acquiring a plurality of chronologically captured observation images of a fertilized egg, and culture environment information regarding the fertilized egg; and

predicting, using the plurality of observation images and the culture environment information, a period of time necessary until the fertilized egg reaches a specified form of development, and a quality of the fertilized egg in the form of development.

(16) An observation system including:

an observation apparatus that includes
an image-capturing section that chronologically captures images of a fertilized egg,
a light source that irradiates light onto the fertilized egg, and
a culture vessel that contains the fertilized egg and a culture solution;

an incubator that accommodates the observation apparatus;

a detector that is capable of detecting a temperature, humidity, and an oxygen concentration in the incubator, pH and an osmotic pressure of the culture solution, concentrations of hormone and a nutrient that are included in the culture solution, a partial pressure of oxygen and a partial pressure of carbon dioxide in the incubator, and illumination intensity of the light source;

an information processing apparatus that includes
an acquisition section that acquires a plurality of observation images chronologically captured by the image-capturing section, and a result of the detection performed by the detector, the plurality of observation images being a plurality of observation images of the fertilized egg, and
a prediction section that predicts, using the plurality of observation images and the result of the detection, a period of time necessary until the fertilized egg reaches a specified form of development, and a quality of the fertilized egg in the form of development; and
a display section that displays the plurality of observation images and a result of the prediction regarding the fertilized egg.
   (17) The observation system according to (16), further including an input section that receives an input of an acquisition request to acquire the fertilized egg cultured for a specified period of time, in which

using the plurality of observation images, the result of the detection, and the acquisition request, the prediction section further generates adjustment parameter information regarding a culture environment of the fertilized egg, and

the information processing apparatus further includes a culture environment controller that controls, according to the adjustment parameter information, at least one of pH of a culture solution used to culture the fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.

### REFERENCE SIGNS LIST

10 observation system
11 incubator
12 observation apparatus
13 humidity-temperature-gas controller
14 detector
15 culture solution adjusting section
16 information processing apparatus
17 display section
18 input section
121 image-capturing section
122 light source
166 prediction section
167 determination section
168 culture environment controller
170 acquisition section
F fertilized egg
W well

1. An information processing apparatus comprising:
an acquisition section that acquires a plurality of chronologically captured observation images of a fertilized egg, and culture environment information regarding the fertilized egg; and
a prediction section that predicts, using the plurality of observation images and the culture environment information, a period of time necessary until the fertilized egg reaches a specified form of development, and a quality of the fertilized egg in the form of development.

2. The information processing apparatus according to claim 1, wherein
the prediction section includes a predictor that is generated using an algorithm in which a plurality of pieces of image data of chronologically captured images of a fertilized egg, and culture environment data of the fertilized egg are training data, and
using the plurality of observation images and the culture environment information, the predictor predicts the necessary period of time and the quality of the fertilized egg in the form of development.

3. The information processing apparatus according to claim 2, wherein
using the plurality of observation images and the culture environment information, the predictor calculates a probability distribution of the period of time necessary until a fertilized egg reaches the form of development, and a quality score that indicates a quality of the fertilized egg.

4. The information processing apparatus according to claim 3, wherein
the predictor calculates, as the quality score, a quality of a fertilized egg after an elapse of a necessary period of time, in which the probability of the fertilized egg reaching the form of development after the elapse of the necessary period of time, is highest in the probability distribution.

5. The information processing apparatus according to claim 3, wherein
the acquisition section further acquires an acquisition request to acquire a fertilized egg cultured for a specified period of time, and
the information processing apparatus further comprises a culture environment controller that controls a culture environment of a fertilized egg according to adjustment parameter information regarding the culture environment, the adjustment parameter information being generated by the predictor using the plurality of observation images, the culture environment information, and the acquisition request.

6. The information processing apparatus according to claim 5, further comprising a determination section that determines whether the quality score for the fertilized egg cultured for the specified period of time is not less than a specified threshold.

7. The information processing apparatus according to claim 6, wherein
the culture environment controller includes a recognizer that is generated using an algorithm in which environment setting data is training data, and
when the determination section has determined that the quality score for the fertilized egg cultured for the specified period of time is not less than the specified threshold, the culture environment controller controls the culture environment of the fertilized egg by applying the adjustment parameter information to the recognizer.

8. The information processing apparatus according to claim 5, wherein
according to the adjustment parameter information, the culture environment controller controls at least one of pH of a culture solution used to culture a fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.

9. The information processing apparatus according to claim 1, wherein
the acquisition section acquires, as the culture environment information, at least one of pH of a culture solution used to culture the fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.

10. The information processing apparatus according to claim 1, wherein
using the plurality of observation images and the culture environment information, the prediction section predicts a period of time necessary until the fertilized egg reaches early blastocyst, blastocyst, or expanded blastocyst.

11. An information processing method comprising:
acquiring a plurality of chronologically captured observation images of a fertilized egg, and culture environment information regarding the fertilized egg; and
predicting, using the plurality of observation images and the culture environment information, a period of time necessary until the fertilized egg reaches a specified form of development, and a quality of the fertilized egg in the form of development.

12. The information processing method according to claim 11, further comprising:
acquiring an acquisition request to acquire the fertilized egg cultured for a specified period of time;
generating, using the plurality of observation images, the culture environment information, and the acquisition request, adjustment parameter information regarding a culture environment of the fertilized egg; and
controlling the culture environment of the fertilized egg according to the adjustment parameter information.

13. The information processing method according to claim 12, wherein
the predicting the period of time necessary until the fertilized egg reaches the specified form of development, and the quality of the fertilized egg in the form of development includes calculating a probability distribution of the period of time necessary until the fertilized egg reaches the form of development, and a quality score that indicates a quality of the fertilized egg.

14. The information processing method according to claim 13, further comprising determining whether the quality score for the fertilized egg cultured for the specified period of time is not less than a specified threshold, wherein
the controlling the culture environment of the fertilized egg includes controlling the culture environment of the fertilized egg according to the adjustment parameter information when the quality score for the fertilized egg cultured for the specified period of time has been determined to not be less than the specified threshold.

15. A program that causes an information processing apparatus to perform a process comprising:
acquiring a plurality of chronologically captured observation images of a fertilized egg, and culture environment information regarding the fertilized egg; and
predicting, using the plurality of observation images and the culture environment information, a period of time necessary until the fertilized egg reaches a specified form of development, and a quality of the fertilized egg in the form of development.

16. An observation system comprising:
an observation apparatus that includes
an image-capturing section that chronologically captures images of a fertilized egg,
a light source that irradiates light onto the fertilized egg, and
a culture vessel that contains the fertilized egg and a culture solution;
an incubator that accommodates the observation apparatus;
a detector that is capable of detecting a temperature, humidity, and an oxygen concentration in the incubator, pH and an osmotic pressure of the culture solution, concentrations of hormone and a nutrient that are included in the culture solution, a partial pressure of oxygen and a partial pressure of carbon dioxide in the incubator, and illumination intensity of the light source;
an information processing apparatus that includes
an acquisition section that acquires a plurality of observation images chronologically captured by the image-capturing section, and a result of the detection performed by the detector, the plurality of observation images being a plurality of observation images of the fertilized egg, and
a prediction section that predicts, using the plurality of observation images and the result of the detection, a period of time necessary until the fertilized egg reaches a specified form of development, and a quality of the fertilized egg in the form of development; and
a display section that displays the plurality of observation images and a result of the prediction regarding the fertilized egg.

17. The observation system according to claim 16, further comprising an input section that receives an input of an acquisition request to acquire the fertilized egg cultured for a specified period of time, wherein
using the plurality of observation images, the result of the detection, and the acquisition request, the prediction section further generates adjustment parameter information regarding a culture environment of the fertilized egg, and
the information processing apparatus further includes a culture environment controller that controls, according to the adjustment parameter information, at least one of pH of a culture solution used to culture the fertilized egg, an osmotic pressure of the culture solution, a concentration of hormone included in the culture solution, a concentration of a nutrient included in the culture solution, a temperature in an incubator used to culture the fertilized egg, humidity in the incubator, an oxygen concentration in the incubator, a partial pressure of oxygen in the incubator, a partial pressure of carbon dioxide in the incubator, or illumination intensity of a light source that irradiates light onto the fertilized egg.
